(19) United States
(12) Patent Application Publication
(10) Pub. No.: US 2011/0098465 A1
(43) Pub. Date: Apr. 28, 2011

(54) Nonflow -Through Apparatus And Method Using Enhanced Flow Mechanisms

(75) Inventors: Carroll Edward Ball, Los Angeles, CA (US); Arkadij M. Elizarov, Woodland Hills, CA (US); Hartmuth C. Kolb, Playa Del Rey, CA (US)

(73) Assignee: SIEMENS MEDICAL SOLUTIONS USA, INC., Malvern, PA (US)

(21) Appl. No.: 12/968,466

(22) Filed: Dec. 15, 2010

Related U.S. Application Data
(62) Division of application No. 12/578,175, filed on Oct. 13, 2009.
(60) Provisional application No. 61/105,247, filed on Oct. 14, 2008.

(51) Int. Cl. C07H 1/00

(52) U.S. Cl. 536/122

(57) ABSTRACT

Methods and apparatus for facilitating the synthesis of compounds in a nonflow-through device are presented. Application of the nonflow-through methods and microfluidic devices to the synthesis of radiolabeled compounds is described. These methods and apparatus enable the introduction of a pressurized gas through a tangential slit into a vortex reactor of the nonflow-through device, while one or more liquids are delivered to the reaction chamber through the same or different inlet ports. The introduction of the pressurized gas produces a cyclonic motion of the mixture within the reactor. Such a mechanism may be used to facilitate the evaporation of various liquids within the reactor at lower temperatures, thus reducing the production of unwanted byproducts that are associated with the use of high temperatures. In addition, thorough mixing of various liquids may be effected rapidly while allowing chemical reactions to take place efficiently within the vortex reactor.

### CLAIM TO PRIORITY

The present application is based on and claims priority to U.S. provisional application No. 61/105,247, filed Oct. 14, 2008, which is hereby incorporated by reference in its entirety herein. The present application is also a divisional application of U.S. Ser. No. 12/578,175, filed on Oct. 13, 2009.

The foregoing application, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF INVENTION

The present invention relates generally to nonflow-through devices and methods for multistep chemical processes using enhanced flow mechanisms and related technologies. More specifically, the present invention relates to methods and microfluidic nonflow-through devices using enhanced flow mechanisms.

### BACKGROUND OF THE INVENTION

Devices, such as microfluidic devices have been used for the preparation of a number of compounds, which may be used in medical imaging applications, such as Positron Emission Tomography (PET) systems, that create images based on the distribution of positron-emitting isotopes in the tissue of a patient. The isotopes are typically administered to a patient by injection of probe molecules that comprise a positron-emitting isotope, such as Fluorine-18, covalently attached to a molecule that is readily metabolized or localized in the body or that chemically binds to receptor sites within the body.

### SUMMARY OF THE INVENTION

One embodiment of the present invention is directed to a nonflow-through apparatus (apparatus) that may be used for carrying out a multistep chemical process. This apparatus comprises a nonflow-through apparatus for carrying out a multistep chemical process and includes a vortex reactor, a volume of which is independent of a volume of one or more incoming reagents/reactants. There are also one or more outlets configured to allow removal of a gas and/or liquids, or a mixture thereof, from the reaction chamber; and one or more inlets configured to deliver a gas and/or a liquid, or a mixture thereof, to the reactor. These are delivered in a direction tangential to the wall of the reactor thereby producing a cyclonic/vortex motion of the gas and/or the liquid, or the mixture thereof, within the reactor to affect the chemical process steps.

This nonflow-through apparatus may be a microfluidic device.

Another embodiment of the present invention is directed toward the apparatus described above, wherein the chemical process steps include concentrating one or more incoming reagents.

Another embodiment of the present invention is directed toward the apparatus described above, wherein the chemical process steps include mixing of the reagents.

Another embodiment of the present invention is directed toward the apparatus described above, wherein the chemical process steps include evaporation of one or more solvents.

Another embodiment of the present invention is directed toward the apparatus described above, wherein the chemical process steps include exchange of one or more solvents.

Another embodiment of the present invention is directed toward the apparatus described above, wherein the chemical process steps include concentrating at least one reaction product.

Another embodiment of the present invention is directed toward the apparatus described above, wherein the chemical process steps are affected by controlling temperature, pressure and a flow rate of a carrier gas.

Another embodiment of the present invention is directed toward the apparatus described above wherein the controlled temperature range is about −78° C. to about 400° C.

Another embodiment of the present invention is directed toward the apparatus described above wherein the chemical process steps are carried out at ambient temperature.

Another embodiment of the present invention is directed toward the apparatus described above wherein the reactor can be pressurized from about −1 atm to 30 atm.

Another embodiment of the present invention is directed toward the apparatus described above, wherein the flow rate of a carrier gas is about 0 to about 100 scfm.

Another embodiment of the present invention is directed toward the apparatus described above, wherein the reagents delivered in low concentration/high volume.

Another embodiment of the present invention is directed toward the apparatus described above, wherein a reaction proceeds in high concentration and low volume.

Another embodiment of the present invention is directed toward the apparatus described above, wherein a concentrated reaction product is eluted in high volume/low concentration.

Another embodiment of the present invention is directed toward the apparatus described above, wherein the reactions are heated while moving by heated incoming carrier gas.

Another embodiment of the present invention is directed toward the apparatus described above, wherein an external source of heat is applied to a bottom part of the vortex reactor to affect the chemical process.

Another embodiment of the present invention is directed toward the apparatus described above, wherein internal volume of the reactor is from about 50 μL to about 10,000 L.

Another embodiment of the present invention is directed toward the apparatus described above, wherein complete evaporation of high boiling solvents is affected.

Another embodiment of the present invention is directed toward the apparatus described above, wherein the high boiling solvents include DMSO, DMF, sulfolane, and water.

Another embodiment of the present invention is directed toward the apparatus described above where is at least two reagents are delivered substantially simultaneously.

Another embodiment of the present invention is directed toward the apparatus described above, wherein the vortex reactor is scalable.

Another embodiment of the present invention is directed toward the apparatus described above, wherein multiple vortex reactors are connected in a variable configuration. The configuration includes sequential, parallel, splitting into multiple paths for creating libraries, or network.

Another embodiment of the present invention is directed toward the apparatus described above, wherein the apparatus is microfluidic and the volume of the reactor is about 5 μL to about 1000 μL.

Another embodiment of the present invention is directed toward the apparatus described above wherein the apparatus is microfluidic and the temperature is about −78° C. to about 400° C.

Another embodiment of the present invention is directed toward the apparatus described above wherein the apparatus is microfluidic and the pressure is about 0 to about 50 psi.

Another embodiment of the present invention is directed toward the apparatus described above wherein the apparatus is microfluidic and the flow rate of a carrier gas is about zero to about 10 scfm.

Another embodiment of the present invention is directed toward the apparatus described above wherein the apparatus is microfluidic and the reaction product is obtained in about 1 to about 60 sec.

Another embodiment of the present invention is directed toward the apparatus described above wherein the apparatus is microfluidic and the chemical process is a radiosynthesis of a radiolabeled compound.

Another embodiment of the present invention is directed to a method for a multistep chemical process effected by the cyclonic motion of a gas and/or a liquid or a mixture thereof in a vortex reactor created by the tangential entry of a pressurized gas into the reactor (the method) and comprising the following steps:

a) delivering the reagents into the reactor;

b) processing the reagent(s) to generate a desired product; and

c) collecting the product.

Another embodiment is directed to the method as described above, wherein at least two reagents are delivered substantially simultaneously.

Another embodiment is directed to the method as described above, wherein one or more reagents delivered in a low concentration are concentrated to a desired volume prior to a reactant's entry.

Another embodiment is directed to the method as described above and further comprising solvent exchange.

Another embodiment is directed to the method as described above, wherein exchanging solvents provides removal of the residual moisture and promotes the drying of a concentrated residue.

Another embodiment is directed to the method as described above further comprising mixing the reagents to effect a chemical reaction by controlling pressure and temperature in the vortex reactor.

Another embodiment is directed to the method as described above further comprising heating or cooling the reagents to effect a chemical reaction by vortex delivery of heated or cooled incoming carrier gas.

Another embodiment is directed to the method as described above further comprising heating the reagents to effect a chemical reaction by an external heat source applied to a bottom part of the reactor.

Another embodiment is directed to the method as described above and eluting the product from the reactor for further processing, wherein the reagents are continuously infused into the reaction chamber. Another embodiment is directed to the method as described above wherein the vortex reactor is microfluidic.

Another embodiment is directed to the method as described above for radiosynthesis of a radiolabeled compound.

Another embodiment is directed to a method of sampling of an ongoing chemical reaction for further analysis by controlling a flow rate of a pressurized gas in a vortex reactor.

Another embodiment is directed a method of sampling of an ongoing chemical reaction for further analysis by controlling a flow rate of a pressurized gas in a vortex reactor wherein the vortex reactor is microfluidic.

Another embodiment is directed to method of sampling of an ongoing chemical reaction for further analysis by controlling a flow rate of a pressurized gas in a vortex reactor wherein the chemical reaction is a radiosynthesis of a radiolabeled compound.

These and other various embodiments of the present invention, together with the organization and manner of operation thereof, will become apparent from the following detailed description when taken in conjunction with the accompanying drawings. The entire disclosures of all patents and references cited throughout this application are incorporated herein by reference in their entirety.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

Embodiments of the invention are described by referring to the attached drawings, in which:

FIG. 1 illustrates exemplary steps for synthesis of a compound using a system according to an embodiment of the present invention;

FIG. 2 illustrates a device in accordance with an embodiment of the present invention;

FIG. 3 illustrates a device in accordance with an embodiment of the present invention; and

FIG. 4 illustrates a device in accordance with an embodiment of the present invention.

### DETAILED DESCRIPTION

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions.

The present invention is directed to a nonflow-through device. This device is used to perform reactions at a desired volume. The nonflow-through apparatus is an apparatus utilized for reactions that are “semi-batch”. This nonflow-through apparatus may be used in microfluidic reactions and for carrying out a multistep chemical process. The nonflow-through device has the advantages of a process that utilizes preferred features of “batch” and “flow” devices and processes.

Specifically, as in the flow process, a first liquid delivered into the reactor while moving around in a cyclonic motion comes in contact with a second liquid which is a flow-through reagent. The first and second liquids contact each other in a flow mode continuously moving in a vortex reactor. This approach enables the key reagents and intermediates to be maintained in a concentrated solution that is moving and does not leave the reactor as in a batch system.

Furthermore, this nonflow-through system allows the reactants to be continuously delivered into the reactor as in the flow-through system. However, they may be further concentrated to a desired volume unlike in the flow-through system. The reactions take place in the vortex reactor in a solution moving rapidly around the walls of the reactor. The product can be collected only once per run as in a batch device. The flow mode allows sampling (aliquoting) of on-going reactions, which is not possible with current batch reactors. It also allows rapid and efficient concentration, solvent exchange and mixing of reagents (even immiscible) while continuously moving around. This system can easily allow the use of intermediates purified by HPLC (current equipment can not accommodate this without major difficulties arising from large volumes of solvent in which the intermediate comes out of HPLC). The features of the vortex reactor allow concurrent introduction of two or more reagents. The presently described nonflow-through vortex reactors allow instantaneous mixing at any scale independent of the volume of the reactor, whereas the mixing rate in both flow-through and batch reactors is inversely proportional to the volume of the reactor. Based on the above-mentioned features, such devices combining both batch and flow-through features allow easy scalability of various chemical protocols and are of great value to research and production applications.

A “microfluidic device” or “microfluidic chip” or “synthesis chip” or “chip” is a unit or device that permits the manipulation and transfer of small amounts of liquid (e.g., microliters or nanoliters) into a substrate comprising micro-channels and micro-compartments. The microfluidic device may be configured to allow the manipulation of liquids, including reagents and solvents, to be transferred or conveyed within the micro-channels and reaction chamber using mechanical or non-mechanical pumps.

The nonflow-through apparatus, for example a microfluidic nonflow-through device, may be constructed using micro-electromechanical fabrication. Alternatively, the nonflow-through devices can be machined using computer numerical control (CNC) techniques. Examples of substrates for forming the device include glass, quartz, silicon, ceramics or polymer. Such polymers may include PMMA (polymethylmethacrylate), PC (polycarbonate), PDMS (polydimethylsiloxane), DCPD (polydicyclopentadiene), PEEK and the like. Such device may comprise columns, pumps, mixers, valves and the like.

Generally, the microfluidic channels or tubes (sometimes referred to as micro-channels or capillaries or conduits) have at least one cross-sectional dimension (e.g., height, width, depth, diameter), which by the way of example, and not by limitation, may range from about 10 μm to about 1,000 μm (microns). The micro-channels permit manipulation of extremely small volumes of liquid, for example on the order of about 1 mL to about 1 μL. The micro reactors may also comprise one or more reservoirs in fluid communication with one or more of the micro-channels, each reservoir having, for example, a volume of about 5 μL to about 1,000 μL.

The microfluidic nonflow-through devices of the present invention offer a variety of advantages over macroscopic reactors that are used for production of compounds, such as radiopharmaceutical compounds. Some examples of advantages of nonflow-through devices, or apparatus, of the present invention include reduced reagent consumption, high concentration of reagents, high surface-to-volume ratios, and improved control over mass and heat transfer.

The reason microfluidic nonflow-through devices is a suitable choice for radiosynthesis is that radiosynthesis involves nanograms of isotope. If the latter is manipulated in any significant volume of solvent, it leads to low concentration and therefore low reaction rate. Meanwhile if it is handled in high concentration (and therefore low volume) most of the isotope would be lost on its way to the microreactor. To claim the benefits of both, one would need to bring the isotope into the reactor in a dilute solution, but to use it in reaction in high concentration. Microfluidic nonflow-through device allows such manipulations.

The nonflow-through devices may also contain multiple reactors with different volumes or features suited for the different steps of a process, where multiple reactors are connected in a number of ways including, but not limited to sequential, parallel, splitting into multiple paths for creating libraries, or a network.

The nonflow-through devices, as described herein, are capable of processing small quantities of molecular probes, as well as expediting chemical processing thereby reducing the overall processing or cycle times, simplifies the chemical processing procedures, and also providing the flexibility to produce a wide range of probes, biomarkers and labeled drugs or drug analogs, inexpensively.

The nonflow-through devices, as described herein, may be used in research and development environments, facilitating the testing and development of new compounds and probes. Co-pending U.S. patent application Ser. Nos. 12/102,822 and 12/176,296, the contents of each of which are hereby incorporated in their entirety by reference, provide descriptive material related to microfluidic devices.

A “radiolabeled compound” is a compound that labels target sites in the body, including, for example, the brain, meaning the compound can be reactive with target sites in the subject.

The term “reactive precursor” or “precursor” refers to an organic or inorganic non-radioactive molecule that is reacted with another reagent typically by nucleophilic substitution, electrophilic substitution, or ionic exchange, to form the product. In case of a radiosynthesis, an organic or inorganic non-radioactive molecule that is reacted with a radioactive isotope, typically by nucleophilic substitution, electrophilic substitution, or ionic exchange, to form the radiopharmaceutical. The chemical nature of the reactive precursor depends upon the physiological process to be studied.

Typically, the reactive precursor is used to produce a radiolabeled compound that selectively labels target sites in the body, including, for example, the brain, meaning the compound can be reactive with target sites in the subject and, where necessary, capable of transport across the blood-brain barrier. Exemplary organic reactive precursors include sugars, amino acids, proteins, nucleosides, nucleotides, small molecule pharmaceuticals, and derivatives thereof. For example, one precursor that may be used in the preparation of 18F-FDG is 1,3,4,6-tetra-O-acetyl-2-O-trifluoromethanesulfonyl-β-D-mannopyranose.

The term “radioactive isotope” refers to isotopes exhibiting radioactive decay (e.g., emitting positrons). Such isotopes are also referred to in the art as radioisotopes or radionuclides. Radioactive isotopes or the correspond ions, such as the fluoride ion, are named herein using various commonly used combinations of the name or symbol of the element and its mass number and are used interchangeably (e.g., 18F, [18F], F-18, [F-18], fluorine-18). Exemplary radioactive isotopes include 124I, 18F, 11C, 13N and 15O, which have half-lives of 4.2 days, 110 minutes, 20 minutes, 10 minutes, and 2 minutes, respectively. The term FLT precursor may be used to refer to “N-dimethoxytrityl-5′-O-dimethoxytrityl-3′-O-nosyl-thymidine” (also known as “BOC—BOC-Nosyl”).

The term “target water” is [18O]H2O after bombardment with high-energy protons in a particle accelerator, such as a cyclotron. Target water contains [18F]fluoride. In one embodiment of the present invention, preparation of target water is contemplated separately from the system disclosed herein. Alternatively, in an embodiment of the present invention, target water is supplied to the system from a cartridge; in another embodiment, from a pre-filled individual vial.

The term “column” means a device that may be used to separate, purify or concentrate reactants or products. Such columns include, but are not limited to, ion exchange and affinity chromatography columns.

A “flow channel” or “channel” means a microfluidic channel through which a fluid, solution, or gas may flow. It is also a channel through which vacuum can be applied. For example, such channels may have a cross section of about 0.1 mm to about 1 mm.

For example, the flow channels of the embodiments of the present invention may also have a cross section dimension in the range of about 0.05 microns (μm) to about 1,000 microns (μm). The particular shape and size of the flow channels depend on the particular application required for the reaction process, including the desired throughput, and may be configured and sized according to the desired application.

The term “vortex reactor” (sometimes referred to as “reactor” or “micro-reactor” or “reaction chamber”) refers to a chamber or a core where the reactions may take place. The reaction chamber may, for example, be cylindrical in shape. The reaction chamber may have one or more micro-channels connected to it that deliver reagents and/or solvents or are designed for product removal (e.g., controlled by on-chip valves, or equivalent devices). For example, the reaction chamber may have a diameter to height ratio of greater than about 0.5 to about 10, or more. By the way of example, and not by limitation, the microfluidic vortex reactor height may be about 25 micrometer (μm) to about 20,000 micrometers (μm).

The term “evaporation” refers to the change in state of solvent from liquid to gas that is usually followed by removal of that gas from the reactor. One method for removing gas is effected by applying a vacuum. Various solvents are evaporated during the synthetic route disclosed herein, such as for example acetonitrile and water. Each solvent, such as acetonitrile and water, may have a different evaporation time and/or temperature.

The term “elution” generally refers to removal of a compound from a particular location. Elution of [18F]fluoride from the ion exchange column refers to the conveyance of [18F]fluoride by the eluting solution from the column to the reaction chamber. Elution of product from the reaction chamber refers to conveyance of the product from the reaction chamber to the off-chip product vial or into the purification system) by, for example, flushing the reaction chamber with a volume of solvent, e.g. water.

The term “cyclonic motion” or “vortex motion” refers to the circular or swirling motion of a gas, liquid and/or a mixture of gas and liquid. For example, such circular motion may occur inside a microfluidic reaction chamber with a circular cross section.

The term “tangential” refers to a tangent line (or simply the tangent) to a curve at a given point is the straight line that “just touches” the curve at that point. As it passes through the point of tangency, the tangent line is “going in the same direction” as the curve, and in this sense it is the best straight-line approximation to the curve at that point. For example, the pressurized gas enters the reactor through a slit in a wall tangentially of this wall to swirl a solution in the reactor along the curve following the direction of a tangent line causing the solution to come into contact with the inner surface of the reactor. In one embodiment, the cylindrical reaction chamber may be comprised of a circular wall and an inlet along a perimeter of the circular wall. An outlet of the first conduit may be in communication with the inlet of the reaction chamber and the outlet of the first conduit is positioned substantially tangential with respect to the perimeter of the circular wall of the reaction chamber. Thus, reagents/gas are introduced tangentially, along the inner portion of the curved wall, creating the vortex effect.

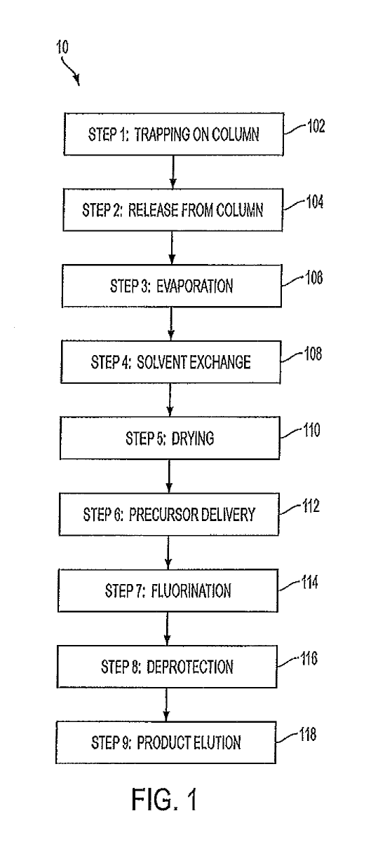

FIG. 1 depicts a series of exemplary steps 10 involved in the synthesis of 18F-labeled compound, such as 18F-FDG, using a nonflow-through device, such as a microfluidic nonflow-through device, taking 18F from cyclotron target water to fluorination reaction in a reactor chip using an ion exchange column.

As shown in FIG. 1, in step 102, Trapping on Column, the target water is typically passed through an ion exchange column (cartridge) to trap the F-18 out of a dilute solution.

In Step 104, Release from Column, the trapped 18F is delivered into a reaction chamber by flushing the ion exchange column using for example, K2CO3 that is released into a concentrated solution that enters the reactor.

After that delivery has taken place, in Step 106 water evaporation takes place. In Step 108, Solvent Exchange, K222/MeCN solution is delivered to the reaction chamber, and in Step 110, Drying, solvents are evaporated, leaving behind a residue containing [18F]KF/K222 complex.

In Step 112, Precursor Delivery, a precursor or reactant (such as Mannose Triflate) is delivered to the reaction chamber. In Step 114, the fluorination reaction takes place. This step may be followed by another drying step (not shown in FIG. 1).

In Step 116, Deprotection, an acid may be delivered to the reaction chamber to remove protecting groups. This step may be followed by another drying step (not shown in FIG. 1). In Step 118, Product elution may need to be performed, requiring water to enter the reaction chamber.

While the exemplary steps shown in FIG. 1 illustrate an overview of the various stages of synthesis that involve the delivery, evaporation, or removal of gas/liquids to and from the reaction chamber, the steps shown in FIG. 1 are not intended to provide an exhaustive or exclusive description of the synthesis process. Accordingly, fewer or more steps may be used to effect synthesis of various compounds such as radiolabeled compounds.

Utilizing a nonflow-through apparatus for carrying out a multistep chemical process enables the conveyance or movement of large volumes of solutions in, out and through the microreactor, thus allowing the removal of reagents which may be concentrated inside the microreactor. The microfluidic nonflow-through apparatus for carrying out a multistep chemical process also facilitates the use of high boiling solvents because the features of the nonflow-through device allows removal of the solvents from the device. Furthermore, the nonflow-through apparatus allows reactions to proceed at lower temperatures, leading to fewer unwanted byproducts.

According to one embodiment of the present invention, by utilizing the nonflow-through apparatus, one or more liquid compounds may enter the reaction chamber, for example, through one or more inlets that are located on the side or the bottom of the reaction chamber, while a carrier gas, such as Nitrogen, may be forced into an inlet opening in such a way that the gas enters the reaction chamber in a direction tangential to the walls of the reaction chamber.

It should be noted that such an opening, while sometimes referred to as a slit or a tangential slit, may comprise any opening such as a port, slit, orifice, vent and combinations thereof that can be configured or structured in such a way to allow the entry of gas and/or liquids into the reaction chamber in a direction that is along the walls (i.e. tangential to walls) of the reaction chamber.

Alternatively, or additionally, either the gas or the liquids may be pulled into the reaction chamber by applying a vacuum to an outlet port, thus producing the negative pressure necessary for introducing the gas and/or liquids into the chamber. The gas that enters the chamber through the opening produces an intimate contact of the liquids with the high velocity gas, mixing with the liquids while entering the reaction chamber with a rotating, cyclonic motion or vortexing motion. Such mixing of the liquids may result in the formation of small droplets. Due to the cyclonic motion, the liquid droplets, along with any solid particles are forced against the interior walls of the reaction chamber under appropriate temperatures. The temperature may be maintained by heating the pressurized gas prior to its entry to the reaction chamber and/or by a heating device that is configured to heat the reaction chamber or a bottom part of it. This step is carried out by reducing or stopping the gas flow and allowing the solution containing the reagents to recede to the lower portion of the reaction chamber, which is heated by an external heating element. At this point, the reaction chamber may be sealed and pressurized.

This heating method is typical for a batch-type reactor.

The above-described droplet formation and cyclonic action may be used to effect mixing of various solutions, reagents and compounds in an efficient manner, thus allowing rapid chemical reactions to take place. In addition, the cyclonic flow of the mixture allows rapid evaporation of the liquids at lower temperatures, with the resultant residue being deposited on the walls of the reaction chamber.

According to an embodiment of the present invention, high boiling solvents, such as DMSO, DMF, sulfolane and solvents with similar properties, may be efficiently and rapidly removed from the reaction chamber at significantly reduced temperatures. In order to remove the deposited solvent or residue, the gas flow may be reduced and a solvent that may be the same or a different solvent, may be introduced to sweep or dissolve the residue from the reaction chamber, which may then be conveyed, for example, into a vial. The above-described steps may be carried out in an automated manner, and repeated as many times as desired.

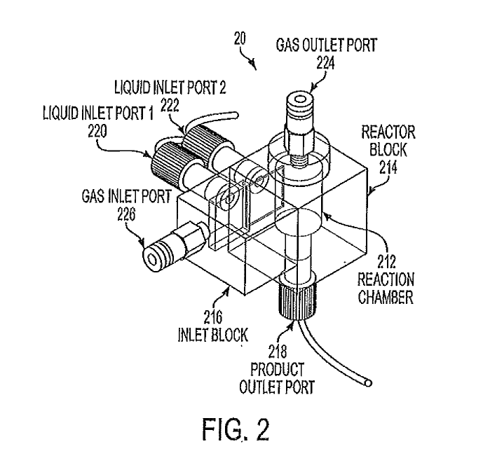

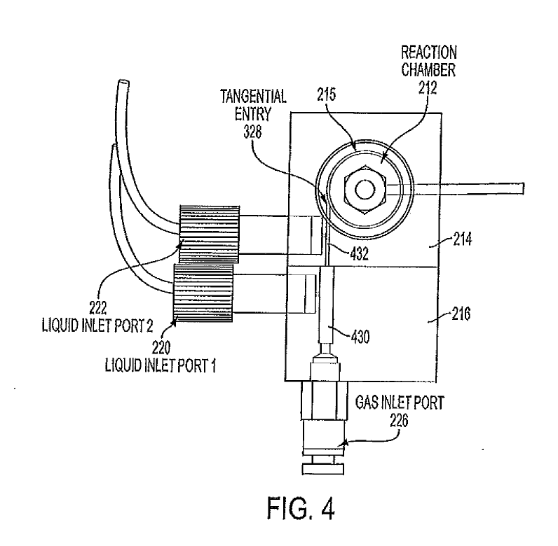

FIG. 2 illustrates an example system 20 that is equipped with a nonflow-through apparatus in accordance with an embodiment of the present invention. This nonflow-through apparatus maybe a microfluidic nonflow-through apparatus for carrying out a multistep chemical process. Reaction chamber 212 may comprise a curved wall 215. As illustrated in FIG. 2, a cylindrical reaction chamber 212 is situated in a reactor block 214. The cylinder comprises side wall (i.e., the curved wall) and perpendicular top and bottom, circular portions (217, 219). The side wall defines the length of the cylindrical reaction chamber and has a length and a width or diameter. (The side wall may comprise an inlet along the curvature of the wall allowing for tangential communication between the conduits (30,32) and the reaction chamber. In the embodiments shown in FIGS. 2-4, the length is greater than the diameter. The system 20 may comprise at least one outlet. The outlet may facilitate the removal of “materials” from the reaction chamber. Such materials may include any materials left over by and/or produced from chemical reactions. This may include reactants, products, by-products, catalysts, liquids, gases, solids, semi-solids, or mixtures thereof, etc. As shown in FIG. 2, in one embodiment, A product outlet port 218 and a gas outlet port 224 allow the removal of various products and gas/vapors from the reaction chamber 212, respectively. In the illustrative embodiment of FIG. 2, a first liquid inlet port 220 (i.e., second inlet port) that is connected to the inlet block 216 may allow liquids to enter the reaction chamber 212, and a second liquid inlet port 222 (i.e., third inlet port) that is connected to the reactor block 214 also allows the entry of liquids into the reaction chamber 212, As shown in FIGS. 2-4, liquid inlet port 1 (second inlet port) may be disposed downstream of the gas inlet port (first inlet port) and upstream of the reaction chamber. Liquid inlet port 2 (third inlet port) may be disposed downstream of the gas inlet port and liquid port 1 but upstream of the reaction chamber. In addition, liquid inlet ports may be disposed substantially perpendicular to the first and second conduits that deliver gas/liquid to the reaction chamber. While the specific configuration of liquid inlet ports of FIG. 2 provides a suitable example for illustrating the underlying concepts of the microfluidic nonflow-through apparatus for carrying out a multistep chemical process. The second and third inlet ports may be in communication with a reagent source such as a gas or liquid and in communication with the reaction chamber either directly or via reactor block or first conduit (FIG. 4).

It is an embodiment of the present invention that these inlet ports (220, 222) may be disposed at different locations and orientations with respect to the reaction chamber 212. In addition, the number of such inlet ports may vary for different configurations. For example, in one embodiment, a single inlet port may be utilized, while in a different configuration, two or more inlet ports may be used. FIG. 2 also illustrates a gas inlet port 226 (i.e., first inlet port) that is connected to the inlet block 216. The gas inlet port 226 may be used to deliver a gas under an appropriate pressure to the reaction chamber 212.

While FIG. 2 shows a reactor block 214 and an intake block 216, it is an embodiment of the present invention that the reactor block 214 and an intake block 216 may be fabricated as a single unit.

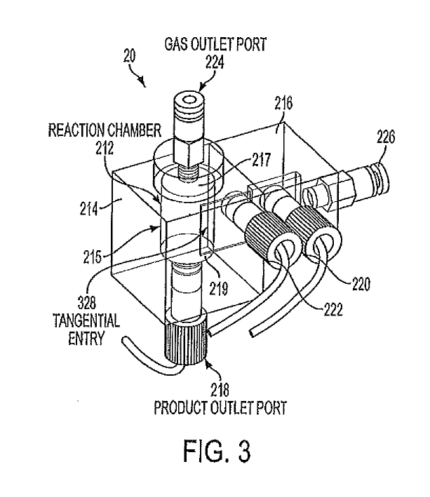

FIG. 3 illustrates a different view of the system 20, where a tangential slit 328 is clearly visible. The tangential slit 328 allows one or more gases and/or a mixture of one or more gases and liquids to enter the reaction chamber 212. The delivery of the one or more gases and/or one or more liquids and/or mixture of one or more gases and one or more liquids to the reaction chamber 212 is also illustrated in FIG. 4 herein. The elements shown in FIG. 2 that are also shown in FIG. 3 are not further described with relation to FIG. 3.

FIG. 4 is a top view of the microfluidic system 20, as described and shown herein. In the illustrative embodiment of FIG. 4, the gas may enter a first conduit 430 and a second conduit 432 before entering the reaction chamber 212 through the tangential slit 328. It is also an embodiment that the liquid from the first inlet port 220 may similarly enter the first conduit 430 and the second conduit 432 before entering the reaction chamber 212 through the tangential slit 328, while the liquid from the second liquid inlet port 222 may enter the second conduit 432 before entering the reaction chamber 212 through the tangential slit 328.

The embodiment illustrated in FIGS. 2 to 4 may be used to deliver a mixture of one or more gases and one or more liquids concurrently to the reaction chamber 212. Alternatively, one or more liquids may enter the reaction chamber 212 first, and then be subjected to the cyclonic motion that is effected due to the subsequent entry of pressurized gas to the reaction chamber 212.

Alternatively one or more of the liquid entry ports may be located at a different location or orientation relative to the gas inlet port 226 and/or the reaction chamber 212. For example, the liquids may enter from the bottom of the reaction chamber 212 through one or more ordinary liquid input ports.

The above-described nonflow-through apparatus may be used in a microfluidic system to efficiently perform the desired chemical reactions by precisely controlling the flow of various solutions into the reaction chamber (shown in FIG. 2 as element 212) while enabling thorough mixing of the reagents. In addition, the nonflow-through apparatus allows rapid and controlled evaporation of solvents at lower temperatures while avoiding the production of unwanted byproducts that are typically associated with the use of high temperatures. Typically, reactions in microfluidic nonflow-through reactors proceed in 1-1000 sec at temperatures of about −78° C. to about 400° C. and pressures of about 0 to 50 psi. The flow rate of the carrier gas, such as nitrogen, is from about 0 to 10 scfm (standard cubic feet per minute).

In one embodiment, switching between a positive and a negative gas pressure allows the user to alternate between the reaction and the evaporation modes of operation. Additionally, or alternatively, a vacuum (not shown) may be applied to effect the conveyance of gas and/or liquids into the reaction chamber (shown in FIG. 2 as element 212), which may be combined with an application of a vacuum to an outlet to maintain and/or facilitate the cyclonic motion and/or evaporation of the mixture.

These and other advantages associated with the use of the nonflow-through apparatus are demonstrated by referring to the previously-described exemplary steps associated with the system of FIG. 1. The following examples demonstrate the application of the nonflow-through apparatus to the various steps associated with the synthesis of a radiolabeled compound in accordance with the various embodiments of the present invention.

### Fluoride Enrichment:

As illustrated in FIG. 1, in Steps 102 and 104, the target water (i.e., [F-18]F— that may be delivered from a cyclotron in approximately 2 mL of dilute solution in [O-18]H₂O) is passed through an ion exchange cartridge to trap the Fluoride that is subsequently eluted with a small volume of K2CO3 solution, for example from about 5 μL to about 100 μL.

In accordance with an embodiment of the present invention, the nonflow-through apparatus can be used to enable complete elution of the trapped Fluoride by allowing a much larger volume of K2CO3 solution, for example from approximately about 400 μL to about 2000 μL to flow through the ion exchange cartridge. According to this embodiment, while the K2CO3 solution flows through the ion exchange cartridge and enters the reaction chamber (212), the solvent may be evaporated in the reaction chamber (212) at a desired rate until complete elution has taken place. The rate of evaporation may be controlled in accordance with the gas pressure entering the reaction chamber (212), as well as the reaction chamber temperature. For example, if the volume of a reaction chamber is about approximately 100 μL and it takes about approximately 400 μL for complete elution, the solvent (i.e., water) may be evaporated at the same rate as the K2CO3 solution entering the reaction chamber 212. Once the entire 400 μL has entered the reaction chamber 212 and solvent has evaporated, a coat of residue is deposited on the walls of the reaction chamber (212).

According to another embodiment of the present invention, the evaporation may be stopped before all of the solvent has completely evaporated, thus facilitating mixing with the reagent that enters the reaction chamber (212) in the next step of the synthesis process.

For example at a temperature 140° C., Pressure 15 psi. Volume of the reactor stays constant at 100 μL. Volume of the reaction mixture varies between 0 and 50 μL depending on the stage of evaporation. The concentration goes from 1 mg/L in the original solution to infinite as the solvent is removed.

### Solvent Exchange:

As illustrated in Step 108 of FIG. 1, in order to solubilize [F-18]Fluoride, a phase transfer reagent such as Kryptofix2.2.2 (K222) may be delivered to the reaction chamber (212). In one embodiment of the present invention, the phase transfer reagent may be introduced as a MeCN solution into the reaction chamber (212) containing [F-18]Fluoride and K2CO3 residue. This task can be readily accomplished using the microfluidic nonflow-through apparatus. However, phase transfer may be conducted more efficiently when water and MeCN are evaporated together as an azeotrope rather allowing sequential evaporation of water and MeCN. Therefore, in accordance with another embodiment of the present invention, phase transfer may be affected by releasing [F-18]Fluoride from the ion exchange column using a mixture of K222 and K2CO3 in a MeCN/H2O solution.

An advantage of the present microfluidic nonflow-through apparatus is that, in accordance with an embodiment of the present invention, the solvents may be delivered to the reaction chamber (212) and evaporated rapidly as more solution is delivered into the reaction chamber (212). In effect, in accordance with the above embodiment, Steps 104 through 110, of FIG. 1, may be combined into a single continuous operation. It should be noted that the evaporation of the solvent using the nonflow-through mechanism may be conducted efficiently so that a single pass is sufficient to remove all the solvents.

However, if necessary, this step may be followed by dry MeCN evaporations using the nonflow-through apparatus. As such, in the event that residual moisture is left in the reactor after evaporation of water, it can be followed by evaporation of dry acetonitrile, which typically removes the last traces of water as an azeotrope.

It is also a feature that continuous azeotropic drying can also be achieved since the residue remains in the reactor while traces of water are removed by evaporation together with acetonitrile which is continuously replenished. This can occur for example at conditions of a temperature 140° C., pressure 15 psi.

### Fluorination:

As illustrated in FIG. 1, Step 112 involves delivering the precursor into the reaction chamber (212) in either a concentrated solution requiring no evaporation, or in a dilute solution, which is concentrated inside the reaction chamber (212).

According to another embodiment of the present invention, by controlling the temperature, pressure and flow rate of the carrier gas, the evaporation of the solution inside the reaction chamber may be suppressed, allowing the reaction to proceed under controlled temperatures and pressures. This task may be carried out according to one of the following two example scenarios. In one embodiment, the precursor solution may be used to sweep the Fluoride/K222/K2CO3 residue from the walls of the reaction chamber (212). This step may be followed by stopping the gas flow and allowing the solution to recede to the lower portion of the reaction chamber, which has a heating element. At this point, the reaction chamber (212) may be sealed and pressurized.

Typically, reactions in the nonflow-through vortex reactors proceed in 1-10000 sec at temperatures of about −78° C. to about 400° C. and pressures of about −1 to 30 atm (the unit of pressure can be also expressed in psi, wherein 1 atm is equal to 14.696 psi). The flow rate of the carrier gas, such as Nitrogen, is from about 0 to 100 scfm (standard cubic feet per minute).

In an alternate embodiment, one or more precursors may be delivered to the reaction chamber (212), while, at the same time, the solvent is evaporated from an atomized mixture inside the reaction chamber (212). As the solution enters the reactor carried by gas into a vortex it instantly turns into an atomized homogeneous state. As the second solution enters, it also turns into the atomized state and the two solutions are instantaneously homogenized because one cannot have two phases in an atomized state (fine mixture of liquid and gas). In this scenario, the reaction is expected to occur rapidly, and be completed by the time the precursor has been added to the reaction chamber (212). Furthermore, the temperature may be maintained by heating the carrier gas and/or heating the reaction chamber. At this stage, the solvent may be completely evaporated, or alternatively, maintained in a solution as the next stage of synthesis commences.

For example, precursor (2 mg) is delivered in 1 mL of MeCN, which is evaporated completely at the end, at temperature of 100° C. and pressure of 3 psi.

### Hydrolysis:

The next step involves the introduction of an acid to remove the protecting groups and yield the radiolabeled compound, for example [F-18]FDG. Similar to the above-described embodiments associated with Fluorination, the Hydrolysis may be carried out in at least one of two exemplary ways.

In one embodiment, the acid may be delivered into the reaction chamber (212) and the reaction may proceed in a solution in a heated recess at the lower portion of the reaction chamber (212).

In an alternate embodiment, the acid may be introduced into a moving solution that is heated by the gas inside the reaction chamber (212). The reaction may or may not be followed by solvent evaporation.

The conditions for this may be for example: 50 μL of 3N HCL is delivered to the reactor and allowed to circulate with gas at temperature of 120° C. and pressure of 5 psi.

Product elution can be performed by water, the volume of which is usually minimal. Meanwhile, if it is desirable for the product to be diluted (e.g. to prevent radiolysis of the product), the elution may be carried out in accordance with another embodiment of the present invention by utilizing the microfluidic nonflow-through apparatus that involves the delivery and evaporation of water, characterized by an elution volume with no upper limits.

The conditions for this may be for example: 200 mL of water is flushed through the reactor at 15 psi while the gas exit port is closed.

Using the nonflow-through apparatus in accordance with the various embodiments of the present invention, allows reagents to be continuously infused into the reaction chamber, while allowing rapid chemical reactions and/or evaporation of solvents to take place. These features facilitate solvent exchange between the various steps involved in the synthesis of radiolabeled compounds in a microreactor.

In accordance with the various embodiments of the present invention, the nonflow-through apparatus allows complete removal of solvents, and especially water, without applying high temperatures that frequently leads to reagent degradation. Additionally, a large volume of reagents may be delivered to the reaction chamber, a feature that is especially beneficial for precursors with low solubility that require large amounts of solvents.

The nonflow-through apparatus allows the evaporation of all solvents, even the ones with high boiling points, such as dimethyl sulfoxide (DMSO) and dimethylformamide (DMF). Due to the high boiling point of these and other similar solvents, their solutions are not generally evaporated in conventional systems.

However, in accordance with an embodiment of the present invention, these and similar high boiling point solvents may be evaporated using the nonflow-through apparatus by utilizing an appropriate gas pressure and a desired temperature of the pressurized gas and/or the reaction chamber. In particular, the high pressure, high velocity gas that enters the reaction chamber causes very fine droplet formation, as well as very efficient mixing and evaporation, every time the reaction chamber contents pass in front of the gas inlet. This procedure is so effective that even a high boiling point solvent, such as DMSO, can be quickly evaporated without any heat addition.

In accordance with another embodiment of the present invention, the use of the nonflow-through apparatus in a microfluidic system allows rapid and efficient mixing of reagents on a molecular level. The mixing may be effected even for reagents that are immiscible. Another feature of the nonflow-through apparatus, which is not available with batch reactors, involves the ability to allow sampling (or aliquotting) of on-going reactions. Thus, by controlling the flow of gas, one may control the level at which the liquid circulates in the reactor. For example, the liquid level may be moved far enough to start reaching the exit port, allowing small fractions of the liquid exit the reactor in a controlled manner. In one embodiment, this task may be accomplished by temporarily reducing the flow of gas, resulting in a slower cyclonic motion, which then allows the removal of a desired portion of the reaction chamber contents through an output port.

Alternatively, in a continuous reaction and concentration operation, a slip stream may be taken from the continuously exiting product stream for intermittent or continuous sampling and analysis.

Another feature of the nonflow-through apparatus allows intermediate purifications by HPLC. As such, the purified intermediate that leaves the HPLC can be fed right back into the reactor in a continuous manner, which is independent of the HPLC solvent mixture. While existing systems are incapable of accommodating this feature due to the large volumes of solvent in which the intermediate comes out of HPLC, the microfluidic nonflow-through apparatus allows continual flow of solvents.

The nonflow-through apparatus in accordance with another embodiment of the present invention, allows high yield reactions to take place at lower temperatures, resulting in fewer byproducts or product decomposition. In addition, it provides the capability to introduce two or more reagents concurrently. These reagents may each enter the reaction chamber separately and then be subjected to the cyclonic motion and atomization that is created by the pressurized gas. Additionally, or alternatively, the two or more reagents may enter the reaction chamber through the same inlet, or through the same tangential inlet as the gas. These and other features of the nonflow-through apparatus allow the synthesis of a larger range of radiolabeled compounds using a microfluidic system.

In one embodiment, the synthetic systems disclosed herein comprise a microfluidic reaction chamber in which, for example, reagents are concentrated, mixed and heated, and solvents are evaporated and exchanged to carry out the desired chemical process.

In another embodiment the evaporation takes place by heating the reaction chamber while flowing an inert gas over the reaction mixture to effect the removal of vapors from the reaction chambers.

In another embodiment, the nonflow-through system comprises one or more reactors connected in a number of ways, which include, but are not limited to sequential, parallel, splitting into multiple paths for creating libraries (where paths split and reconnect), or network and having the capability to simultaneously vary one or more of the process conditions, which include, but are not limited to flow rates, pressure, temperature and feed composition.

In another embodiment, the nonflow-through apparatus is scalable. The nonflow-through apparatus may perform equally in volume ranges from about 5 μL to about 10,000 L.

More specifically, the volumes of reactions performed with nonflow through apparatus may be increased from initial volumes; but the results of the reactions using the higher volumes are proportional to the results of the initial volumes. Thus, the results of small volume reactions, for example from about 5 μL to about 500 μL apply to reactions performed using higher volumes for example from about 5 mL to 10,000 L.

Generally, embodiments of the present invention are directed to systems methods and apparatus for synthesis of radiolabeled compounds and to improve efficiency of radiosynthesis using microfluidic devices.

Some examples of the radiolabeled compounds that may be prepared according to one or more embodiments of the present invention include compounds selected from the group of 2-deoxy-2-[18F]fluoro-D-glucose ([18F]FDG), 6-[18F]fluoro-L-3,4-dihydroxyphenylalanine ([18F]FDOPA), 6-[18F]fluoro-L-meta-tyrosine ([18F]FMT), 9-[4-[18F]fluoro-3-(hydroxymethyl)butyl]guanine ([18F]FHBG), 9-[(3-[18F]fluoro-1-hydroxy-2-propoxy)methyl]guanine ([18F]FHPG), 3-(2′-[18F]fluoroethyl)spiperone ([18F]FESP), 3′-deoxy-3-[18F]fluorothymidine ([18F]FLT), 4-[18F]fluoro-N-[2-[1-(2-methoxyphenyl)-1-piperazinyl]ethyl]-N-2-pyridinyl-benzamide ([18F]p-MPPF), 2-(1-{6-[(2-[18F]fluoroethyl)(methyl)amino]-2-naphthyl}ethylidine)malononitrile ([18F]FDDNP), 2-[18F]fluoro-α-methyltyrosine, [18F]fluoromisonidazole ([18F]FMISO) and 5-[18F]fluoro-2′-deoxyuridine ([18F]FdUrd).

One embodiment of the present invention relates to a method for a radiosynthesis of a radiolabeled compound. This method includes introducing one or more reagents into the nonflow-through device. The nonflow-through device comprising a vortex reaction chamber, one or more outlets connected to the reaction chamber to allow removal of gas and/or liquids from the reaction chamber, and one or more inlets connected to the reaction chamber to allow delivery of a gas and/or a liquid, or a mixture thereof, to the reaction chamber in a direction tangential to the walls of the reaction chamber. The method further comprises processing the reagent(s) to generate the radiolabeled compound, and collecting the radiolabeled compound.

Furthermore, one or more inlets may be, for example, a port, slit, orifice, vent and combination thereof.

In another embodiment of the present invention, the entry of a mixture of pressurized gas and liquids into the reaction chamber produces a cyclonic motion within the reaction chamber. As used herein, a “liquid” may be a solvent that is introduced into the reaction chamber, or a “liquid” may be a solution that comprises a solvent and a substrate or reagent.

In yet another embodiment of the present invention, two or more liquids are mixed within the reaction chamber as a result of the cyclonic motion.

In yet another embodiment of the present invention, a chemical reaction is effected within the reaction chamber.

In yet another embodiment of the present invention, a liquid within the reaction chamber is evaporated.

In yet another embodiment of the present invention, a residue is deposited on the walls of the reaction chamber after a substantially complete evaporation of the liquid. The residue may comprise a reagent that is used in the reaction, or upon completion of the desired reaction, the residue may comprise the product that is obtained from the reaction.

In yet another embodiment of the present invention, at least one of the gas and the liquid is heated prior to entry to the reaction chamber to effect evaporation of the liquid inside the reaction chamber.

In yet another embodiment of the present invention, a mixture of pressurized gas and liquids is continuously delivered to the reaction chamber.

In yet another embodiment of the present invention, the continuous delivery of the pressurized gas is necessary to promote the cyclonic motion in the vortex reactor.

In yet another embodiment of the present invention, the continuous delivery of the pressurized gas is independent of the delivery of incoming liquids.

In yet another embodiment of the present invention, the method further comprises a heater for heating the reaction chamber.

In yet another embodiment of the present invention, the nonflow-through device, may be a microfluidic device that further comprises one or more liquid inlets to allow entry of liquids into the reaction chamber.

In yet another embodiment of the present invention, entry of a liquid through a first liquid inlet and entry of a pressurized gas through a second inlet in a direction tangential to the walls of the reaction chamber produces a cyclonic motion of a mixture of gas and liquids within the reaction chamber. Under such conditions, two or more liquids may be mixed within the reaction chamber as a result of the cyclonic motion.

In yet another embodiment of the present invention, a chemical reaction is effected within the reaction chamber.

In yet another embodiment of the present invention, a liquid within the reaction chamber is evaporated.

In yet another embodiment of the present invention, one or more liquids are continuously delivered to the reaction chamber.

In yet another embodiment of the present invention, the microfluidic device is used to effect aliquotting the contents of the reaction chamber.

In yet another embodiment of the present invention, the reaction chamber may be configured with a separation or chromatographic system to allow intermediate purification of the desired product by high-performance liquid chromatography (HPLC).

Yet another embodiment of the present invention, relates to a microfluidic apparatus, comprising a microfluidic reaction chamber, one or more outlets configured to allow removal of gas and/or liquids from the reaction chamber and one or more inlets configured to deliver a gas and/or a liquid, or a mixture thereof, to the reaction chamber in a direction tangential to the walls of the reaction chamber.

The inlet and the outlet may be, for example, configured in the reaction chamber to convey a liquid within the reaction chamber to be removed from the reaction chamber.

While the foregoing description has been primarily described using the embodiment that utilizes one inlet slit (shown as 328 herein) for the delivery of gas, it is understood that according to another embodiment of the present invention, the nonflow-through apparatus may be implemented using two or more inlet slits. One or more of the slits may be used to deliver the gas and/or one or more liquids to the reaction chamber (212).

In accordance with a further embodiment, one or more solutions may be introduced simultaneously with the gas through the same slit.

According to yet another embodiment, one or more liquids may be delivered to the reaction chamber through one or more slits, while the gas is introduced to the chamber using a different slit.

In still another embodiment, more than one slit may be used to deliver the gas into the reaction chamber (212).

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments.

The embodiments discussed herein were chosen and described in order to explain the principles and the nature of various embodiments and its practical application to enable one skilled in the art to utilize the present invention in various embodiments and with various modifications as are suited to the particular use contemplated. The features of the embodiments described herein may be combined in all possible combinations of methods, apparatus, modules, systems and computer program products.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for a multistep chemical process effected by the cyclonic motion of a gas and/or a liquid or a mixture thereof in a vortex reactor created by the tangential entry of a pressurized gas into the reactor and comprising the following steps:
a) delivering the reagents into the reactor;
b) processing the reagent(s) to generate a desired product; and
c) collecting the product.

2. The method of claim 1, wherein at least two reagents are delivered substantially simultaneously.

3. The method of claim 1, wherein one or more reagents delivered in a low concentration are concentrated to a desired volume prior to a reactant's entry.

4. The method of claim 1, wherein one or more reagents delivered in a low concentration are concentrated to a desired volume during reactant's entry.

5. The method of claim 1, further comprising solvent exchange.

6. The method of claim 5, wherein exchanging solvents provides removal of the residual moisture and promotes the drying of a concentrated residue.

7. The method of claim 1 further comprising mixing the reagents to effect a chemical reaction by controlling pressure and temperature in the vortex reactor.

8. The method of claim 1 further comprising heating or cooling the reagents to effect a chemical reaction by vortex delivery of heated or cooled incoming carrier gas.

9. The method of claim 1 further comprising heating the reagents to effect a chemical reaction by an external heat source applied to a bottom part of the reactor.

10. The method of claim 1 further comprising:
eluting the product from the reactor for further processing,
wherein the reagents are continuously infused into the reaction chamber.

11. The method of claim 1, wherein the vortex reactor is microfluidic.

12. The method of claim 1 for a radiosynthesis of a radiolabeled compound.

13. A method of sampling of an ongoing chemical reaction for further analysis by controlling a flow rate of a pressurized gas in a vortex reactor.

14. The method of claim 13, wherein the vortex reactor is microfluidic.

15. The method of claim 13, wherein the chemical reaction is a radiosynthesis of a radiolabeled compound.

16. A method for carrying out a chemical reaction in an apparatus comprising:
a reaction chamber having a curved wall; and
a gas inlet upstream of the reaction chamber;
the method comprising:
providing a gas into the reaction chamber, substantially tangential with respect to the curved wall;
providing a liquid reagent to the reaction chamber,
wherein the tangential entry of the gas creates a vortex of the gas and liquid within the reaction chamber.

17. The method of claim 16, wherein the liquid reagent is introduced upstream of the reaction chamber but downstream of the gas source.

18. The method of claim 17, wherein the liquid is introduced substantially perpendicular with respect to a direction of travel of the gas from the gas inlet toward the reaction chamber.

19. The method of claim 17, wherein the gas moves the liquid into the reaction chamber.

20. The method of claim 19, wherein the liquid is introduced into the reaction chamber, substantially tangential with respect to the curved wall.

21. The method of claim 16, wherein the vortex effect facilitates generation of products of a chemical reaction.

22. The method of claim 21, further comprising the step of releasing the products from the reaction chamber.

23. The method of claim 22 further comprising the step of releasing gas from the reaction chamber.

24. The method of claim 16, wherein the apparatus further comprises a plurality of liquid reagent sources upstream of the reaction chamber and downstream of the gas inlet, wherein the method further comprises providing liquid reagent from each of the plurality of sources and mixing the liquid reagent with gas from the gas source, within the reaction chamber.

25. The method of claim 16, further comprising evaporating at least one solvent within the reaction chamber.

26. The method of claim 16, further comprising controlling the pressure of the reaction at about −1.0 atm. to about +30.0 atm.

27. The method of claim 16, further comprising providing the gas to the reaction chamber at a rate of about 0 to about 100 scfm.

28. The method of claim 16, further comprising heating the gas to a temperature greater than that of the reagent.

29. The method of claim 24, wherein each of the plurality of reagent sources comprise a different reagent and wherein the reagents are delivered substantially simultaneously.

30. The method of claim 16, further comprising providing about 1 mL to about 1,000 μL of reagent.

31. The method of claim 16, comprising providing the gas at a velocity sufficient to break-up the liquid into a plurality of droplets.

32. The method of claim 21, further comprising reducing velocity of the gas and introducing a solvent into the reaction chamber, wherein the solvent sweeps any reaction residue from the reaction chamber walls.

33. The method of claim 31, wherein the droplets are microscopic in size.

34. The method of claim 16, comprising heating the reaction chamber with a heat source and stopping the gas flow and allowing the solution containing the reagents to recede to the lower portion of the reaction chamber, near the heat source.

35. The method of claim 16, comprising changing the temperature of the carrier gas to control the temperature within the reaction chamber.
